(19) United States
(12) Patent Application Publication
(10) Pub. No.: US 2007/0177741 A1
(43) Pub. Date: Aug. 2, 2007

(54) Batteryless noise canceling headphones, audio device and methods for use therewith

(76) Inventor: Matthew Ross Williamson, Austin, TX (US)

Correspondence Address: GARLICK HARRISON & MARKISON, P.O. BOX 160727, AUSTIN, TX, 78716-0727, US

(21) Appl. No.: 11/344,272

(22) Filed: Jan. 31, 2006

(51) Int. Cl. A61F 11/06; H04R 1/10; G10K 11/16

(52) U.S. Cl. 381/71.6; 381/74

(57) ABSTRACT

Batteryless noise canceling headphones include a first earpiece having a first speaker for providing a first audio output to a first ear of a user in response to a first audio output signal, and having a first microphone element for converting a first ambient audio input at the first ear into a first ambient audio input signal in response to a bias voltage. A connector receives the bias voltage from an audio device, receives the first audio output signal from the audio device, and provides the first ambient audio signal to the audio device.

### TECHNICAL FIELD OF THE INVENTION

The present invention relates to noise canceling headphones and related methods used for devices such as audio devices.

### DESCRIPTION OF RELATED ART

Headphones are used in a wide variety of audio equipment including portable or or non-portable devices. Such portable devices include compact disk (CD) players, MP3 players, digital video disk (DVD) players, amplitude modulation/frequency modulation (AM/FM) radios, cellular telephones, laptop computers, multifunction devices, etc. Examples of non-portable devices include stereo systems, video systems, desktop computers, keyboards and other electronic musical instruments, etc. Each of these devices typically include one or more integrated circuits to provide the functionality of the device. Headphones can be used in a variety of places that include environments where the ambient noise is distracting or otherwise detracts from a user's experience in listening to the audio content produced by the equipment.

Noise canceling headphones exist that use a microphone to produce an ambient noise signal that can be mixed with the audio content so as to cancel or reduce the user's hearing of the ambient sound. These devices employ active circuitry that require a source of power to operate that adds to the cost of operation, and the cost, complexity and weight of the device.

The need exists for noise canceling headphones that can be implemented efficiently and in a cost effective manner.

### BRIEF DESCRIPTION OF THE SEVERAL VIEWS OF THE DRAWINGS

FIG. 1 presents a pictorial diagram representation of a handheld audio system in accordance with an embodiment of the present invention.

FIG. 2 presents a pictorial diagram representation of a multifunction portable device in accordance with an embodiment of the present invention.

FIG. 3 presents a pictorial diagram representation of a CD player in accordance with an embodiment of the present invention.

FIG. 4 presents a pictorial diagram representation of a computer in accordance with an embodiment of the present invention.

FIG. 5 presents a block diagram representation of an audio device in accordance with an embodiment of the present invention.

FIG. 6 presents a block diagram representation of a noise cancellation module in accordance with an embodiment of the present invention.

FIG. 7 presents a block diagram representation of an audio device in accordance with an embodiment of the present invention.

FIG. 8 presents a pictorial view of headphones in accordance with an embodiment of the present invention.

FIG. 9 presents a pictorial view of headphones in accordance with an embodiment of the present invention.

FIG. 10 presents a pictorial view of headphones in accordance with an embodiment of the present invention.

FIG. 11 presents a pictorial view of a connector in accordance with an embodiment of the present invention.

FIG. 12 presents a pictorial/schematic view of a connector and jack in accordance with an embodiment of the present invention.

FIG. 13 presents a pictorial/schematic view of a connector and jack in accordance with an embodiment of the present invention.

FIG. 14 presents a flowchart representation of a method in accordance with an embodiment of the present invention.

FIG. 15 presents a flowchart representation of a method in accordance with an embodiment of the present invention.

FIG. 16 presents a flowchart representation of a method in accordance with an embodiment of the present invention.

FIG. 17 presents a flowchart representation of a method in accordance with an embodiment of the present invention.

### DETAILED DESCRIPTION OF THE INVENTION INCLUDING THE PRESENTLY PREFERRED EMBODIMENTS

The embodiments of the present invention yield several advantages over the prior art. Batteryless noise canceling headphones obtain power from the audio device to which they are connected to avoid the added weight and complexity of having on-board batteries.

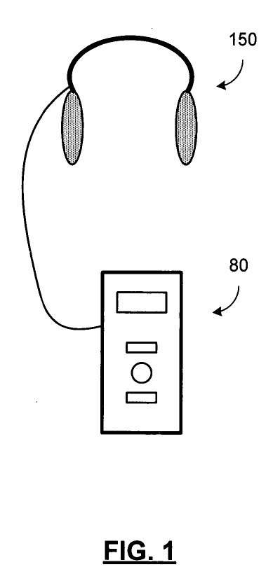

FIG. 1 presents a pictorial diagram of a handheld audio system in accordance with an embodiment of the present invention. In particular, a handheld audio system 80 is shown that receives a radio signal. In an embodiment of the present invention, the radio signal includes one or more of a broadcast frequency modulated (FM) radio signal, an in-band on-channel (IBOC) digital radio signal, a Bluetooth signal, a broadcast amplitude modulated (AM) radio signal, a broadcast satellite radio signal, and a broadcast cable signal.

In operation, the handheld audio system 80 produces an audio output by means of batteryless noise canceling headphones 150 that reduce the ambient noise heard by the user. In addition to producing an audio output from the received radio signal, the handheld audio system 80 can optionally process stored MP3 files, stored WMA files, and/or other stored digital audio files to produce the audio output. The batteryless noise canceling headphones implement features and functions in accordance with one or more embodiments of the present invention that are discussed herein.

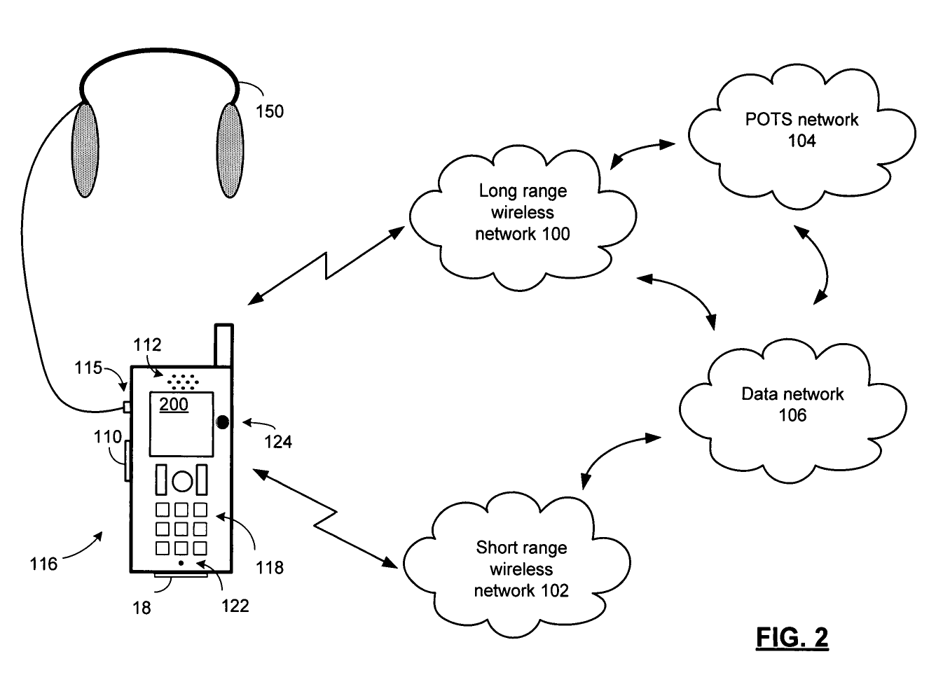

FIG. 2 presents a pictorial diagram representation of a multifunction portable device in accordance with an embodiment of the present invention. In particular, a multifunction portable device 116 communicates over a long range wireless network 100 that is operably coupled to the plain old telephone service (POTS) network 104 and data network 106. Multifunction portable device 116 can further communicate over short range wireless network 102 to data network 106. In an embodiment of the present invention, long range network 100 includes a wireless telephone network such as cellular, personal communications service (PCS), general packet radio service (GPRS), global system for mobile communications (GSM), and integrated digital enhanced network (iDEN) or other wireless communications network capable of sending and receiving telephone calls. Further, data network 106 includes the Internet and short range wireless network 102 includes an access point that communicates with the multifunction portable device 116 via a radio frequency communications link such as 802.11x, Wimax, a wireless local area network connection of other communications link. In this fashion, multifunction portable device 116 can place and receive telephone calls, text messages such as emails, short message service (SMS) messages, pages and other data messages that may include multimedia attachments such as documents, audio files, video files, images and other graphics and further can access streaming audio content, either alone or in conjunction with streaming video.

Multifunction portable device 116 optionally includes a camera 124 for capturing still and/or video images, removable memory card 100 for providing additional memory and removable storage, and host interface 18 for uploading and downloading information directly to a host device such as a computer.

Multifunction portable device 116 includes internal audio input device such as microphone 122 and internal audio output device such as speaker 112 The user interface of multifunction portable device 116 includes a keypad 118 and a display device, such as touch screen 200, for displaying graphics and text, and for providing an additional touch sensitive interface with soft keys and/or graphics input and or handwriting recognition. Multifunction portable device 116 includes batteryless noise canceling headphones 150 that can optionally be connected via headphone jack 115 in accordance with one or more embodiments of the present invention that are discussed herein

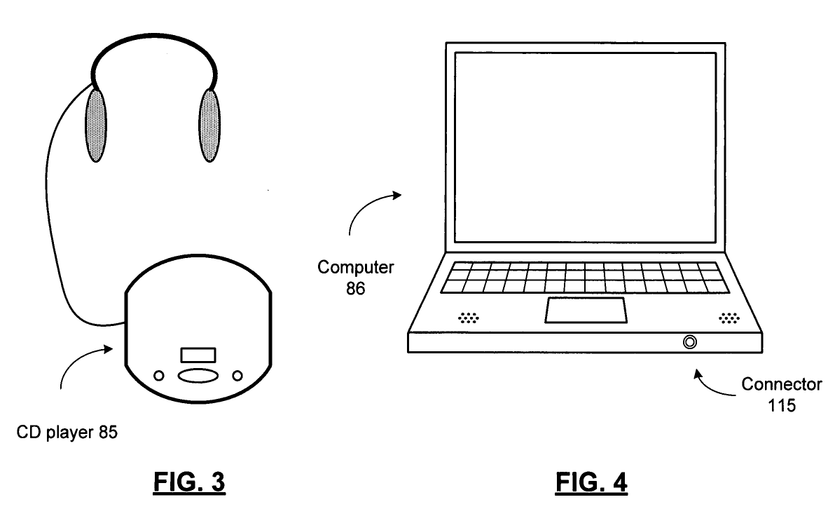

FIG. 3-4 present pictorial diagram representations of a CD player and computer in accordance with an embodiment of the present invention. While the preceding disclosure has been directed to batteryless noise canceling headphones 150 used in conjunction with handheld audio system 80 and/or multifunction portable device 116, in an embodiment of the present invention, these headphones may be used in conjunction with in a wide variety of audio devices such as compact disk player 86, in computer 86 when coupled to an input jack such as connector 115 or in a variety of other portable and non-portable electronic devices that provide an audio output.

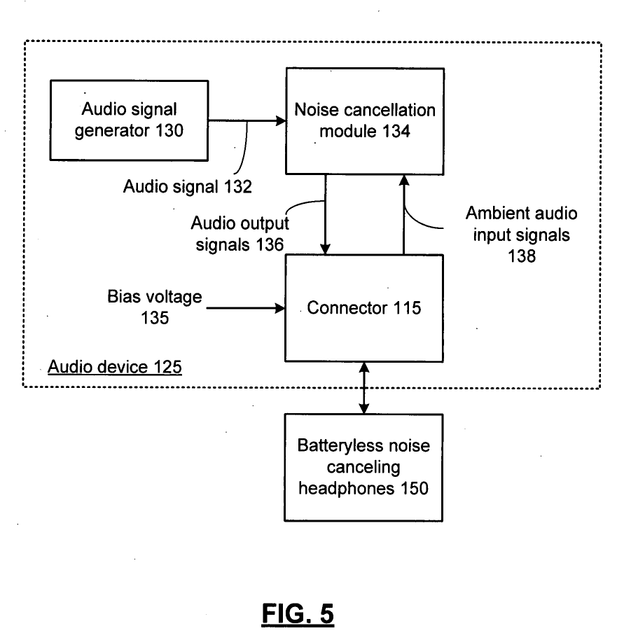

FIG. 5 presents a block diagram representation of an audio device in accordance with an embodiment of the present invention. Audio device 125 includes an audio signal generator 130 for generating an audio signal 132 that can be based on a broadcast signal such as a radio signal, a recorded signal such as from a stored audio file or from a CD or DVD, or a streaming signal such as a streaming audio or video signal. Noise cancellation module 134 processes one or more ambient audio input signals 138 from batteryless noise canceling headphones 150 into one or more cancellation signals that are mixed with the channels of the audio signal 132 with the cancellation signals to produce a first audio output signal 136. Audio device 125 further includes a connector 115, operably coupled to the noise cancellation module 134 that, when the batteryless noise canceling headphones 150 are connected, provide the audio output signals 136 and a bias voltage 135 to the batteryless noise canceling headphones to, respectively, drive the speakers and to provide power to one or more microphones that are integrated into the headphones. In addition, connector 115 receives the ambient audio input signals 138 from the microphones of batteryless noise canceling headphones 150.

In an embodiment of the present invention, the bias voltage 135 is generated from the battery, power supply or other power source of audio device 125. This eliminates the need for the headphones to having to rely upon their own source of power, such as their own batteries.

In an embodiment of the present invention, the audio signal 132 includes a first channel signal and a second channel signal such as right and left channel signals of a stereo signal and the batteryless noise canceling headphones 150 include a single microphone or otherwise generate a single ambient audio input signal 138 that is processed by noise cancellation module 134 into a single cancellation signal. In this embodiment, the noise cancellation module 134 is further operable to mix the first channel signal with the cancellation signal to produce a first audio output signal and to mix the second channel signal with the cancellation signal to produce a second audio output signal. The connector 115 is operable to provide the first and second audio output signal to the batteryless noise canceling headphones 150.

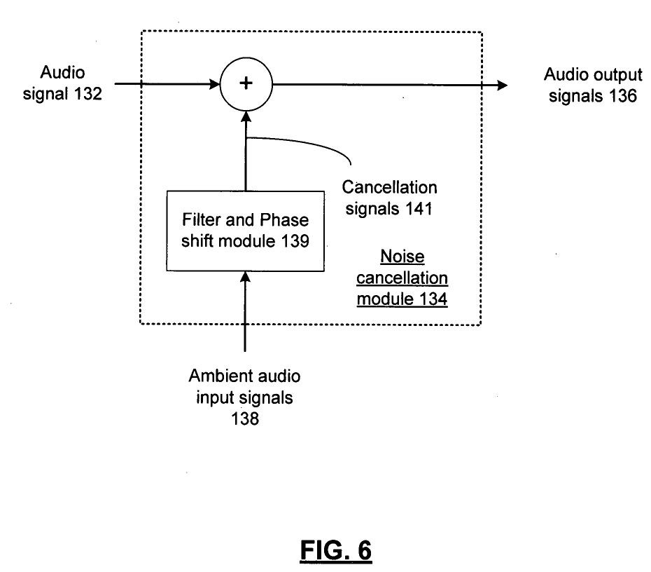

FIG. 6 presents a block diagram representation of a noise cancellation module in accordance with an embodiment of the present invention. In an embodiment of the present invention, the audio signal 132 includes a first channel signal and a second channel signal such as right and left channel signals of a stereo signal and the batteryless noise canceling headphones 150 include a two or more microphones or otherwise generate a two ambient audio input signals 138 that are processed by noise cancellation module 134 into noise cancellation signals 141 that include corresponding first and second cancellation signals. In this embodiment, the noise cancellation module 134 is further operable to mix the first channel signal with the first cancellation signal to produce the first audio output signal and to mix the second channel signal with the second cancellation signal to produce a second audio output signal. The connector 115 is operable for providing the first and second audio output signals to the batteryless noise canceling headphones 150 and to receive the first and second ambient audio input signal from the microphones.

In a preferred embodiment of the present invention, the ambient audio input signals 138 are generated by two microphones of batteryless noise canceling headphones 150 that are each located in close proximity to an ear of the user. Noise cancellation module 134 generates the cancellation signals 141 using filter and phase shift module 139 that shifts the phase of these signals so that, when provided to the right and left speakers of the headphones, they generate audio outputs that add destructively with the ambient noise within the ear canal (are of substantially equal amplitude and substantially opposite phase) so as to be heard at reduced level by the user or not at all. In an embodiment of the present invention, filter and phase shift module 139 also low-pass filters the ambient audio input signals 138 prior to creating the cancellation signals 141, because high frequencies are difficult to cancel due to their smaller wavelength. However, since the ears' reception of high frequency sounds is more highly directional and higher frequency sounds are easier to block with pass noise filtration such as the earpiece enclosure of the headphones, the overall effect can be a substantial improvement in overall perceived noise by the user across a wide audio spectrum.

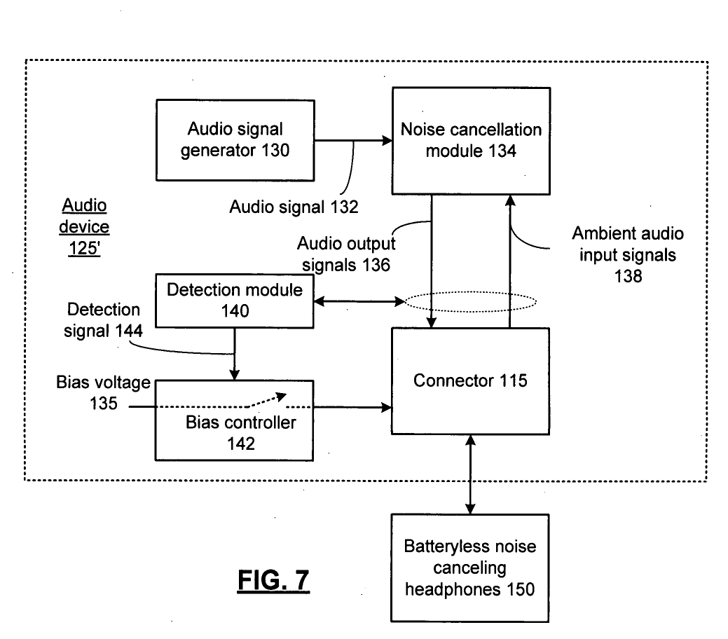

FIG. 7 presents a block diagram representation of an audio device in accordance with an embodiment of the present invention. In particular an audio device 125′ is provided wherein connector 115 is further operable for providing the audio output signals 136 to non-noise canceling headphones (such as traditional headphones or earbuds) when the non-noise canceling headphones are connected thereto. In addition to components of audio device 125, audio device 125′ includes a device detection module 140 operably coupled to the connector 115, for generating a detection signal 144 when the non-noise canceling headphones are connected to the connector and a bias controller 142 for decoupling the bias voltage to the connector 115 in response to the detection signal 144.

In an embodiment of the present invention, detection module 140 detects whether a device is connected to the conductors of connector 115 by measuring the impedance, voltage or current from one of more of these conductors and generates a bi-state detection signal 144 in response. In particular, detection module 140 detects whether the microphone or microphones are coupled to the conductors that couple the bias voltage 135 to batteryless noise canceling headphones 150, and if so, generates a detection signal 144 having a state that commands bias controller 142 to couple the bias voltage 135 to connector 115. If no microphone is connected as detected by a short circuit, open circuit or other voltage, impedance or current that indicates that a microphone is not connected, detection module 140 generates a detection signal 144 having a state that commands bias controller 142 to not couple the bias voltage 135 to connector 115. While bias controller. 142 is shown as including a single-pole single-throw switch for this purpose, this function may be accomplished by a transistor, relay, or other circuit for selectively supplying the bias voltage 135 to connector 115 when batteryless noise canceling headphones are detected as being coupled to connector 115 and for not supplying the bias voltage 135 to connector 115 when non-noise canceling headphones, are connected.

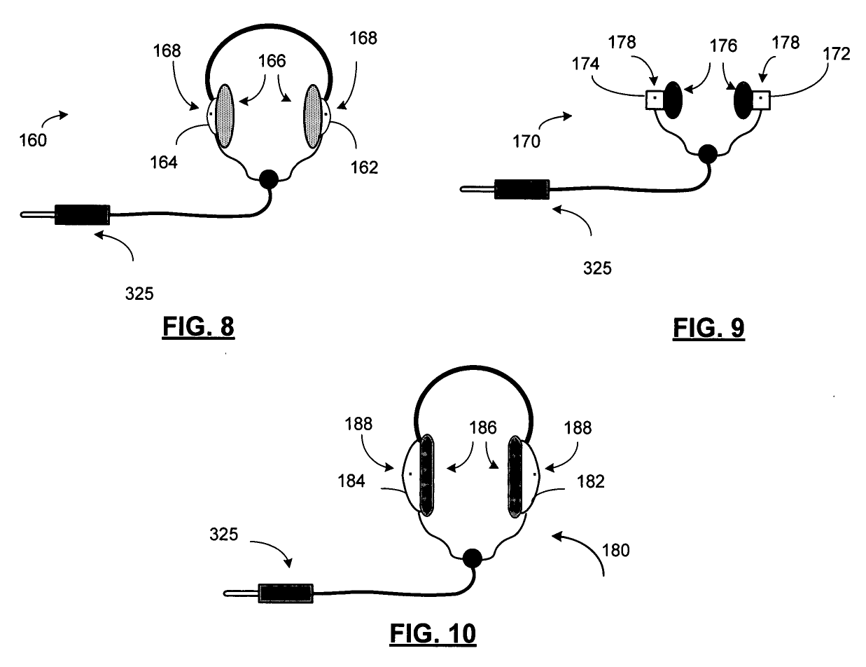

FIG. 8 presents a pictorial view of headphones in accordance with an embodiment of the present invention. In particular, batteryless noise canceling headphones 160 are presented that include earpieces 162 and 164 with speakers 166 and microphone elements 168 that are integrated into supra-aural enclosures that rest upon the user's ears in operation.

FIG. 9 presents a pictorial view of headphones in accordance with an embodiment of the present invention. In particular, batteryless noise canceling headphones 170 are presented that include earpieces 172 and 174 with speakers 176 and microphone elements 178 that are integrated into earbud enclosures that rest within the user's ears in operation.

FIG. 10 presents a pictorial view of headphones in accordance with an embodiment of the present invention. In particular, batteryless noise canceling headphones 180 are presented that include earpieces 182 and 184 with speakers 186 and microphone elements 188 that are integrated into circum-aural enclosures that cover the user's ears in operation.

In an embodiment of the present invention, batteryless noise canceling headphones 150, 160, 170 and 180 each include at least one earpiece, such as earpieces 162, 172, 182, having a first speaker, such as speakers 166, 176, 186, for providing a first audio output to a first ear of a user in response to a first audio output signal, and having a first microphone, such as microphones 168, 178, 188, for converting a first ambient audio input at the first ear into a first ambient audio input signal in response to a bias voltage, such as bias voltage 135. Each design includes a second earpiece, such as earpieces 164, 174, 184, operably coupled to the connector 325, the second earpiece having a second speaker, such as speakers 166, 176, 186, for providing a second audio output to a second ear of a user in response to a second audio output signal and a second microphone, such as microphones 168, 178, 188, for converting a second ambient audio input at the second ear into a second ambient audio input signal in response to the bias voltage. Each design further includes a connector 325 operably coupled to the first speaker and the first microphone, for connecting to an audio device, such as audio devices 125 and/or 125′. In an embodiment, the connector receives the bias voltage from the audio device, receives the first audio output signal from the audio device, and provides the first ambient audio signal to the audio device and is further operable for receiving the second audio output signal from the audio device and for providing the second ambient audio signal to the audio device.

While FIGS. 8-10 illustrate batteryless noise canceling headphones with two earpieces, a design with a single earpiece may likewise be implemented within the broad scope of the present invention.

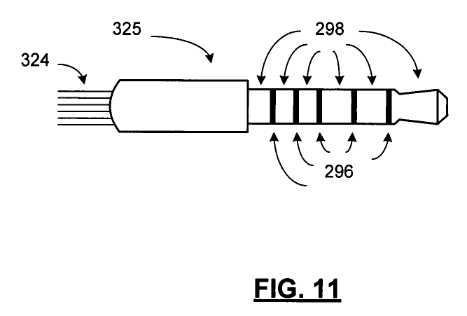

FIG. 11 presents a pictorial view of a connector in accordance with an embodiment of the present invention. In particular, a connector 325 is shown as a plug connector for coupling batteryless noise canceling headphones 150, 160, 170 and/or 180 to a jack connector such as connector 115. In an embodiment of the present invention, the connector 325 conforms to the dimensions of a miniature stereo phone jack commonly used for standard headphone connections. Connector 325 includes a plurality of conductors 298 that are separated by insulating elements 296 in a configuration that allows the right channel audio signal, left channel audio signal and ground to be coupled to the headphones via wires 324 in a fashion that is compatible with existing miniature stereo phone jacks, but that includes additional conductors 298 for coupling the bias voltage 135 and the ambient audio input signals 138 to and from the headphones via wires 324.

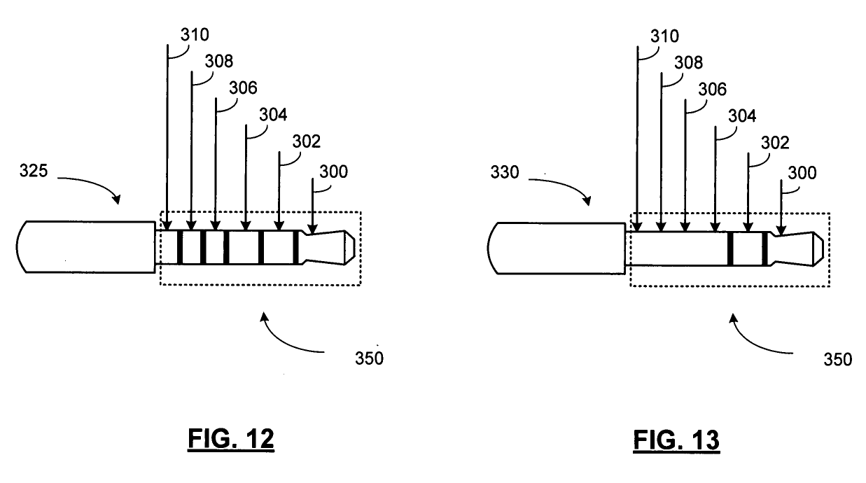

FIG. 12 presents a pictorial/schematic view of a connector and jack in accordance with an embodiment of the present invention. In particular, plug 325 is shown schematically connected to a jack 350, such as connector 115, that includes conductors 300, 302, 304, 306, 308 and 310 for making contact with conductors 296 of plug 325. In an embodiment of the present invention, conductors 304, 302 and 300 couple ground, and audio output signals 132, such as right and left audio outputs, to right and left speakers of the batteryless noise canceling headphones in the standard way. Conductors 306, 308 and 310 couple the bias voltage, such as bias voltage 135, to the microphones of the headphones and receive a right and left ambient audio input signals such as ambient audio input signals 138.

In an embodiment of the present invention, separate conductors are provided on the plug 325 and jack 350 to connect separate ambient audio input and audio output signal grounds to the batteryless noise canceling headphones to provide additional signal isolation between the ambient audio input signals 138 and the audio output signals 132. This can be accomplished by adding an additional conductor to plug 325 and jack 350 or by using a monaural ambient audio input signal 138 and using one of the conductors 306, 308, or 310 for ground instead of a second ambient audio input signal.

FIG. 13 presents a pictorial/schematic view of a connector and jack in accordance with an embodiment of the present invention. In particular, a plug 330 from standard stereo non-noise canceling headphones is shown coupled to jack 350. In this configuration, conductors 304, 302 and 300 couple ground and right and left audio output signals to the headphones in a standard way. Conductors 306, 308 and 310 are unused by the audio device, such as audio device 125′ and, in particular, the bias voltage 135 is decoupled from the jack 350 so as to not provide bias voltage back to an audio output channel or ground.

While the connectors 325, 350 and 115 are described in conjunction with a miniature phone plug that is compatible with existing stereo headphone connections, other plug and jack combinations may likewise be employed within the broad scope of the present invention, in particular, connectors 325, 350 and 115 can be of the male or female, monaural or stereo varieties. Connectors 325, 350 and 115 can be implemented in a standard configuration such as a ¼″ phone connector or subminiature phone connector, RCA phone connector, 8-pin ham microphone connector, coaxial connector of N size, H size or other size, an S-video connector, a banana jack connector, a PL-259 connector, an F connector, a BNC connector or other plug or jack connector, either standard or non-standard that can be coupled and decoupled.

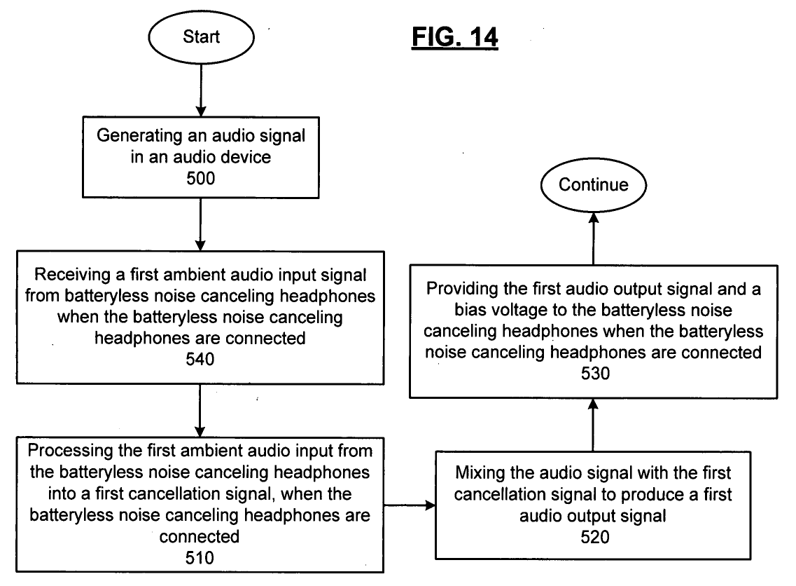

FIG. 14 presents a flowchart representation of a method in accordance with an embodiment of the present invention. In particular, a method is presented for use in conjunction with one or more of the features and functions presented in association with the embodiments presented in association with FIGS. 1-13. In step 500 an audio signal is generated in an audio device. In step 510 a first ambient audio input signal is received from batteryless noise canceling headphones when the batteryless noise canceling headphones are connected to the audio device. In step 520, the first ambient audio input signal is processed from the batteryless noise canceling headphones into a first cancellation signal when the batteryless noise canceling headphones are connected to the audio device. In step 530, the audio signal is mixed with the first cancellation signal to produce a first audio output signal. In step 540, the first audio output signal and a bias voltage are provided to the batteryless noise canceling headphones when the batteryless noise canceling headphones are connected to the audio device.

In an embodiment of the present invention, the audio signal includes one of: a broadcast signal, a recorded signal, and a streaming signal.

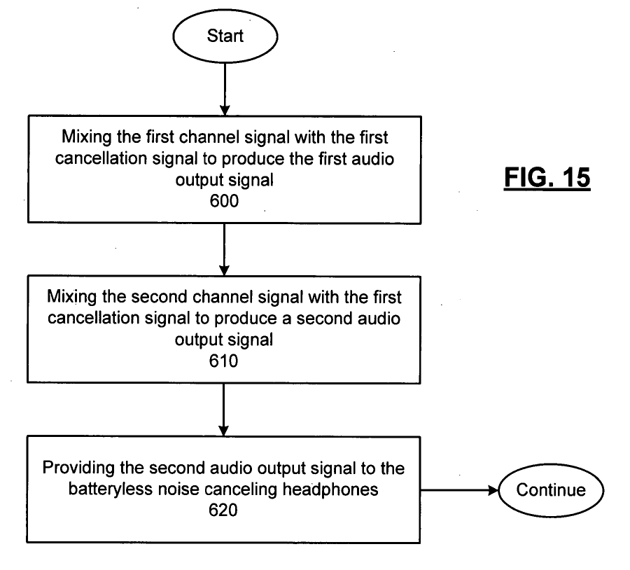

FIG. 15 presents a flowchart representation of a method in accordance with an embodiment of the present invention. A method is presented for use in conjunction with one or more of the features and functions presented in association with the embodiments presented in association with FIGS. 1-13, and particular for use with step 520 of FIG. 14. In step 600, the first channel signal is mixed with the with the first cancellation signal to produce the first audio output signal. In step 610, the second channel signal is mixed with the first cancellation signal to produce a second audio output signal. In step 620, second audio output signal is provided to the batteryless noise canceling headphones.

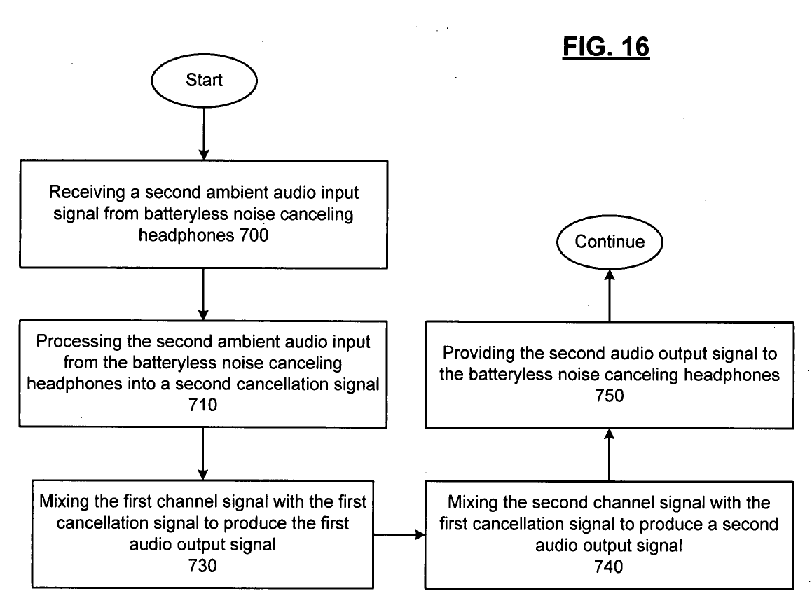

FIG. 16 presents a flowchart representation of a method in accordance with an embodiment of the present invention. A method is presented for use in conjunction with one or more of the features and functions presented in association with the embodiments presented in association with FIGS. 1-13, and particular for use with step 520 of FIG. 14. In step 700 a second ambient audio input signal is received from the batteryless noise canceling headphones. In step 710, the second ambient audio input signal from the batteryless noise canceling headphones is processed into a second cancellation signal. In step 720, the first channel signal is mixed with the first cancellation signal to produce a first audio output signal. In step 730, the second channel signal is mixed with the second cancellation signal to produce a second audio output signal. In step 740, the second audio output signal is provided to the batteryless noise canceling headphones.

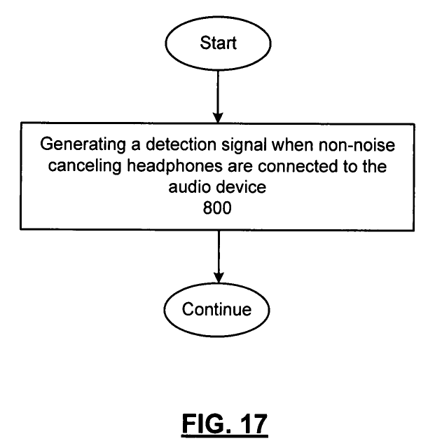

FIG. 17 presents a flowchart representation of a method in accordance with an embodiment of the present invention. In particular, a method is presented for use in conjunction with one or more of the features and functions presented in association with the embodiments presented in association with FIGS. 1-16. In step 800, a detection signal is generated when non-noise canceling headphones are connected to the audio device.

While the operation of the modules of audio device 125 and 125′ are described in terms of circuit implementations, various elements and modules presented in the embodiments described herein can also be implemented with a processor and memory module. It should also be noted that the software implementations of the present invention can be stored on a tangible storage medium such as a magnetic or optical disk, read-only memory or random access memory and also be produced as an article of manufacture.

In an embodiment of the present invention, the processor can be a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on operational instructions. The memory may be a single memory device or a plurality of memory devices. Such a memory device may be a read-only memory, random access memory, volatile memory, non-volatile memory, static memory, dynamic memory, flash memory, cache memory, and/or any device that stores digital information. Note that when the processing module implements one or more of its functions via a state machine, analog circuitry, digital circuitry, and/or logic circuitry, the memory storing the corresponding operational instructions may be embedded within, or external to, the circuitry comprising the state machine, analog circuitry, digital circuitry, and/or logic circuitry.

In preferred embodiments, the various circuit components are implemented using 0.35 micron or smaller CMOS technology. Provided however that other circuit technologies, both integrated or non-integrated, may be used within the broad scope of the present invention.

As one of ordinary skill in the art will appreciate, the term “substantially” or “approximately”, as may be used herein, provides an industry-accepted tolerance to its corresponding term and/or relativity between items. Such an industry-accepted tolerance ranges from less than one percent to twenty percent and corresponds to, but is not limited to, component values, integrated circuit process variations, temperature variations, rise and fall times, and/or thermal noise. Such relativity between items ranges from a difference of a few percent to magnitude differences. As one of ordinary skill in the art will further appreciate, the term “operably coupled”, as may be used herein, includes direct coupling and indirect coupling via another component, element, circuit, or module where, for indirect coupling, the intervening component, element, circuit, or module does not modify the information of a signal but may adjust its current level, voltage level, and/or power level. As one of ordinary skill in the art will also appreciate, inferred coupling (i.e., where one element is coupled to another element by inference) includes direct and indirect coupling between two elements in the same manner as “operably coupled”. As one of ordinary skill in the art will further appreciate, the term “compares favorably”, as may be used herein, indicates that a comparison between two or more elements, items, signals, etc., provides a desired relationship. For example, when the desired relationship is that signal 1 has a greater magnitude than signal 2, a favorable comparison may be achieved when the magnitude of signal 1 is greater than that of signal 2 or when the magnitude of signal 2 is less than that of signal 1.

As the term module is used in the description of the various embodiments of the present invention, a module includes a functional block that is implemented in hardware, software, and/or firmware that performs one or module functions such as the processing of an input signal to produce an output signal. As used herein, a module may contain submodules that themselves are modules.

Thus, there has been described herein an apparatus and method, as well as several embodiments including a preferred embodiment, for implementing batteryless noise canceling headphones. Various embodiments of the present invention herein-described have features that distinguish the present invention from the prior art.

It will be apparent to those skilled in the art that the disclosed invention may be modified in numerous ways and may assume many embodiments other than the preferred forms specifically set out and described above. Accordingly, it is intended by the appended claims to cover all modifications of the invention which fall within the true spirit and scope of the invention.

What is claimed is:

1. An audio device comprising:
an audio signal generator for generating an audio signal;
a noise cancellation module for processing a first ambient audio input signal from batteryless noise canceling headphones into a first cancellation signal and for mixing the audio signal with the first cancellation signal to produce a first audio output signal;
a connector, operably coupled to the noise cancellation module, for providing the first audio output signal and a bias voltage to the batteryless noise canceling headphones and for receiving the first ambient audio input signal from the batteryless noise canceling headphones when the batteryless noise canceling headphones are connected thereto.

2. The audio device of claim 1 wherein the audio signal includes a first channel signal and a second channel signal, wherein the noise cancellation module is further operable for mixing the first channel signal with the first cancellation signal to produce a first audio output signal and for mixing the second channel signal with the first cancellation signal to produce a second audio output signal, and wherein the connector is operable for providing the second audio output signal to the batteryless noise canceling headphones.

3. The audio device of claim 1 wherein the audio signal includes a first channel signal and a second channel signal, wherein the noise cancellation module is further operable for processing a second ambient audio input signal from the batteryless noise canceling headphones into a second cancellation signal, for mixing the first channel signal with the first cancellation signal to produce the first audio output signal and for mixing the second channel signal with the second cancellation signal to produce a second audio output signal, and wherein the connector is operable for providing the second audio output signal to the batteryless noise canceling headphones and to receive the second ambient audio input signal from the batteryless noise canceling headphones.

4. The audio device of claim 1 wherein the audio signal includes one of: a broadcast signal, a recorded signal, and a streaming signal.

5. The audio device of claim 1 wherein the connector is further operable for providing the first audio output to non-noise canceling headphones, when the non-noise canceling headphones are connected thereto.

6. The audio device of claim 5 further comprising:
a device detection module, operably coupled to the connector, for generating a detection signal in a first state when the non-noise canceling headphones are connected to the connector; and
a bias controller, operably coupled to the connector and the detection module, for decoupling the bias voltage from the connector in response to the first state of the detection signal.

7. The audio device of claim 5 further comprising:
a device detection module, operably coupled to the connector, for generating a detection signal in a second state when the noise canceling headphones are connected to the connector; and
a bias controller, operably coupled to the connector and the detection module, for coupling the bias voltage to the connector in response to the second state of the detection signal.

8. The audio device of claim 1 wherein the batteryless noise canceling headphones includes a first earpiece having one of: an earbud enclosure, a supra-aural enclosure and a circum-aural enclosure.

9. Batteryless noise canceling headphones comprising:
a first earpiece having a first speaker for providing a first audio output to a first ear of a user in response to a first audio output signal, and having a first microphone element for converting a first ambient audio input at the first ear into a first ambient audio input signal in response to a bias voltage;
a connector, operably coupled to the first speaker and the first microphone, for connecting to an audio device, the connector for receiving the bias voltage from the audio device, for receiving the first audio output signal from the audio device, and for providing the first ambient audio signal to the audio device.

10. The batteryless noise canceling headphones of claim 9 wherein the first earpiece includes one of: an earbud enclosure, a supra-aural enclosure and a circum-aural enclosure.

11. The batteryless noise canceling headphones of claim 9 further comprising:
a second earpiece, operably coupled to the connector, the second earpiece having a second speaker for providing a second audio output to a second ear of a user in response to a second audio output signal;
wherein the connector is further operable for receiving the second audio output signal from the audio device.

12. The batteryless noise canceling headphones of claim 11 wherein the second earpiece includes a second microphone element for converting a second ambient audio input at the second ear into a second ambient audio input signal in response to the bias voltage, and wherein the connector is further operable for providing the second ambient audio signal to the audio device.

13. A method comprising:
generating an audio signal in an audio device;
receiving a first ambient audio input signal from batteryless noise canceling headphones when the batteryless noise canceling headphones are connected to the audio device;
processing the first ambient audio input signal from the batteryless noise canceling headphones into a first cancellation signal when the batteryless noise canceling headphones are connected to the audio device;
mixing the audio signal with the first cancellation signal to produce a first audio output signal; and
providing the first audio output signal and a bias voltage to the batteryless noise canceling headphones when the batteryless noise canceling headphones are connected to the audio device.

14. The method of claim 13 wherein the audio signal includes a first channel signal and a second channel signal, wherein the step of processing the first ambient audio input signal includes:
mixing the first channel signal with the first cancellation signal to produce the first audio output signal;
mixing the second channel signal with the first cancellation signal to produce a second audio output signal; and
providing the second audio output signal to the batteryless noise canceling headphones.

15. The method of claim 13 wherein the audio signal includes a first channel signal and a second channel signal, wherein the step of processing the first ambient audio input signal includes:
receiving a second ambient audio input signal from the batteryless noise canceling headphones;
processing the second ambient audio input signal from the batteryless noise canceling headphones into a second cancellation signal;
mixing the first channel signal with the first cancellation signal to produce a first audio output signal;
mixing the second channel signal with the second cancellation signal to produce a second audio output signal; and
providing the second audio output signal to the batteryless noise canceling headphones.

16. The method of claim 13 wherein the audio signal includes one of: a broadcast signal, a recorded signal, and a streaming signal.

17. The method of claim 13 further comprising:
generating a detection signal when non-noise canceling headphones are connected to the audio device.
